(19) United States
(12) Patent Application Publication
(10) Pub. No.: US 2024/0360419 A1
(43) Pub. Date: Oct. 31, 2024

(54) METHOD FOR PRODUCING VASCULARIZED RESPIRATORY ORGANOID FUSED WITH BLOOD VESSEL ORGANOID

(71) Applicant: THE CATHOLIC UNIVERSITY OF KOREA INDUSTRY-ACADEMIC COOPERATION FOUNDATION, Seoul (KR)

(72) Inventors: Sung Won KIM, Seoul (KR); Ayoung JO, Seoul (KR); Min Jae LIM, Gyeonggi-do (KR); Mi Hyun LIM, Gyeonggi-do (KR)

(21) Appl. No.: 18/647,191

(22) Filed: Apr. 26, 2024

(30) Foreign Application Priority Data
Apr. 27, 2023 (KR) 10-2023-0055710
Apr. 16, 2024 (KR) 10-2024-0050963

(51) Int. Cl. C12N 5/071; C12N 5/00

(52) U.S. Cl. CPC C12N 5/0691; C12N 5/0062; C12N 5/0688; C12N 5/0697; C12N 2502/27; C12N 2502/28; C12N 2506/45; C12N 2513/00

(57) ABSTRACT

Disclosed is a method of producing vascularized respiratory (airway and alveolar) organoids using blood vessel organoids. More particularly, vascularized respiratory (airway and alveolar) organoids with a vascular network are produced by fusing respiratory (airway and alveolar) organoids with blood vessel organoids to induce vascularization. The vascularized respiratory (airway and alveolar) organoids can have functions very similar to the human respiratory whole region, so they are expected to be transplanted into humans to treat respiratory related diseases or be used as an in vitro respiratory whole region model.

### CROSS-REFERENCE TO RELATED APPLICATION

This application claims priority to Korean Patent Application No. 10-2023-0055710, filed on Apr. 27, 2023, and Korean Patent Application No. 10-2024-0050963, filed on Apr. 16, 2024, the disclosures of which are each incorporated herein by reference in their entireties.

### BACKGROUND OF THE INVENTION

### 1. Field of the Invention

The present disclosure relates to a method of producing a vascularized respiratory (airway and alveolar) organoid fused with blood vessel organoids, etc.

### 2. Description of the Related Art

In the process of developing new drugs, animal testing on animals similar to humans is required prior to clinical trials. Animals such as mice and rabbits are used in animal testing, so the ethics thereof are problematic. In addition, since animal testing models have different genetic or biological characteristics from the human body, existing drug tests have limitations in the reliability of drug test responses, such as side effects that were not found in animal testing being found in humans.

Several methods are being studied to complement the problems in a new drug development process. However, an existing 2D cell line culture method has the disadvantage that it is difficult to reproduce the unique properties and characteristics of the tissue. As an alternative, xenograft models derived from cancer patients are used, but they have the disadvantage of being unsuitable for large-scale drug screening. Therefore, research on experimental methods using organoids has been actively conducted recently.

Organoids are organ analogs created by three-dimensionally cultivating or recombining stem cells. They are derived from various body organs and comprise stem cells, forming aggregates that can differentiate into cell bodies. In addition, organoids derived from each organ in the body have a structure very similar to the organ from which they are derived, so they can be used as a model that can replace cell and animal experiments.

Meanwhile, to reproduce an accurate drug response or disease phenotype as a human organ-simulating model, mature organoids similar to those of an adult are required, but most current organoid induction and two-dimensional culture methods have limitations in that organoids are necrotic or exist in an immature state before differentiation into mature organoids. Therefore, it can be said that the formation of a vascular network is very essential for better function as mature organoids and the maturation thereof.

As a result of intensive research to increase the maturity of organoids, the present inventors developed vascularized respiratory (airway and alveolar) organoids with a vascular network by fusing respiratory (airway and alveolar) organoids with blood vessel organoids, thus completing the present disclosure.

### RELATED ART DOCUMENT

### Patent Document

Korean Patent Application Publication No. 10-2023-0007766

### SUMMARY OF THE INVENTION

Therefore, the present disclosure has been made in view of the above problems, and it is an object of the present disclosure to provide a method of producing vascularized respiratory organoids, the method comprising:
(S1) gelating blood vessel organoids and respiratory organoids dispensed into one well; and
(S2) culturing and fusing the gelated blood vessel organoids and respiratory organoids of step (S1) in a medium for blood vessel organoid maturation.

It is another object of the present disclosure to provide vascularized respiratory organoids produced by the method.

It is yet another object of the present disclosure to provide a vascularized respiratory organoid chip comprising an organoid culture chamber loaded with the vascularized respiratory organoids.

However, the technical problem to be achieved by the present disclosure is not limited to the problems mentioned above, and other problems not mentioned can be clearly understood by those skilled in the art from the following description.

In accordance with the present disclosure, the above and other objects can be accomplished by the provision of a method of producing a vascularized respiratory organoid, the method comprising:
(S1) gelating a blood vessel organoid and respiratory organoid dispensed into one well; and
(S2) culturing and fusing the gelated blood vessel organoid and respiratory organoid of (S1) in a medium for blood vessel organoid maturation.

In an embodiment of the present disclosure, the blood vessel organoid may be derived from induced pluripotent stem cells (iPSC), without being limited thereto.

In an embodiment of the present disclosure, the induced pluripotent stem cells may be derived from one or more cells selected from the group consisting of umbilical cord blood, alveolar epithelial cells and basal cells, without being limited thereto.

In an embodiment of the present disclosure, the blood vessel organoid may express one or more of CD31 (cluster of differentiation 31) and SMA (smooth muscle actin).

In an embodiment of the present disclosure, the respiratory organoid may be one of an alveolar organoid and an airway organoid, without being limited thereto.

In an embodiment of the present disclosure, when the respiratory organoid is an airway organoid, the method may further comprise (S0) stabilizing an airway organoid and a blood vessel organoid in a medium for blood vessel organoid maturation, without being limited thereto.

In an embodiment of the present disclosure, in step (S1), a ratio of the respiratory organoid: the blood vessel organoid may be 1: (1 to 5), without being limited thereto.

In an embodiment of the present disclosure, step (S1) may be performed at 30° C. to 40° C. for 10 minutes to 2 hours, without being limited thereto.

In an embodiment of the present disclosure, in step (S2), the culturing may be performed for 1 day to 15 days, without being limited thereto.

In accordance with another aspect of the present disclosure, there is provided a vascularized respiratory organoid, produced by the method.

In an embodiment of the present disclosure, the respiratory organoid may be one of an alveolar organoid and an airway organoid, without being limited thereto.

In an embodiment of the present disclosure, the vascularized respiratory organoid may have a structure wherein a blood vessel organoid surrounds a respiratory organoid, without being limited thereto.

In an embodiment of the present disclosure, the vascularized respiratory organoid may have a morphological feature wherein a new blood vessel is generated and spreads out, without being limited thereto.

In an embodiment of the present disclosure, when the vascularized respiratory organoid is a vascularized alveolar organoid, the vascularized alveolar organoid may express one or more of CD31 (cluster of differentiation 31) and SMA (smooth muscle actin), without being limited thereto.

In an embodiment of the present disclosure, when the vascularized respiratory organoid is a vascularized airway organoid, the vascularized respiratory organoid may express one or more of CD31 (cluster of differentiation 31) and tubulin.

In accordance with still another aspect of the present disclosure, there is provided a vascularized respiratory organoid chip, comprising an organoid culture chamber loaded with the vascularized respiratory organoid.

In an embodiment of the present disclosure, the organoid culture chamber may comprise a medium for blood vessel organoid maturation, without being limited thereto.

In accordance with still another aspect of the present disclosure, there is provided a kit for producing vascularized respiratory organoids, the kit comprising:
(a) a composition for producing blood vessel organoids; and
(b) a composition for producing respiratory organoids,
wherein each of the compositions comprises one selected from a group consisting of components below:
(a) umbilical cord blood, alveolar epithelial cells, and basal cells; and
(b) alveolar epithelial tissue and basal cells.

In accordance with still another aspect of the present disclosure, there is provided use of (a) a composition for producing blood vessel organoids and (b) a composition for producing respiratory organoids to produce vascularized respiratory organoids.

In accordance with yet another aspect of the present disclosure, there is provided use of (a) a composition for producing blood vessel organoids and (b) a composition for producing respiratory organoids to produce a vascularized respiratory organoid.

### BRIEF DESCRIPTION OF THE DRAWINGS

The above and other objects, features and other advantages of the present disclosure will be more clearly understood from the following detailed description taken in conjunction with the accompanying drawings, in which:

FIG. 1 illustrates a protocol for producing blood vessel organoids;

FIGS. 2A and 2B illustrate induced pluripotent stem cells observed at each step during a production process of blood vessel organoids using alveolar epithelial cells, and induced pluripotent stem cells derived from basal cells, respectively;

FIGS. 3A and 3B illustrate results of confirming CD31 and SMA, which are vessel markers, in blood vessel organoids using alveolar epithelial cells, and induced pluripotent stem cells derived from basal cells, respectively;

FIGS. 4A and 4B illustrate the generation and expansion of new blood vessels from blood vessel organoids using alveolar epithelial cells, or induced pluripotent stem cells derived from basal cells, after gelation;

FIGS. 5A and 5B illustrate the shapes of lung-blood vessel fusion organoids and respiratory tract-blood vessel fusion organoids, respectively; and

FIGS. 6A and 6B illustrate the morphologies and expression markers of lung-blood vessel fusion organoids and respiratory tract-blood vessel fusion organoids confirmed using immunofluorescence staining.

### DETAILED DESCRIPTION OF THE INVENTION

As a result of intensive research to increase the maturity of organoids, the present inventors developed vascularized respiratory organoids with a vascular network, i.e., lung-blood vessel fusion organoids and respiratory tract-blood vessel fusion organoids, by fusing respiratory (airway and alveolar) organoids with blood vessel organoids, thus completing the present disclosure.

The vascularized airway organoids of the present disclosure are characterized by a structural arrangement wherein blood vessel organoids surround airway organoids. As the blood vessel organoids are gelated, morphological characteristics wherein new blood vessels are created and spread out are observed. It was confirmed that this can act as a microenvironment that can express an environment similar to an actual tissue. Accordingly, the vascularized airway organoids of the present disclosure did not only imitate specific tissues of the human body, but also exhibited significantly increased similarity to tissues due to the development of a microenvironment that can express an environment similar to actual tissues. In addition, it was confirmed that the maturity of organoids was improved, and, since the organoids are cell aggregates much larger than 2D cells, highly functional airway organoids that can solve the problem of cell necrosis caused by nutrient and oxygen deficiency in the center can be provided.

The present disclosure provides a method of producing vascularized respiratory organoids, the method comprising the following steps:
(S1) gelating blood vessel organoids and respiratory organoids dispensed into one well; and
(S2) culturing and fusing the gelated blood vessel organoids and respiratory organoids of step (S1) in a medium for blood vessel organoid maturation.

Accordingly, the present disclosure provides a method of producing vascularized respiratory (airway and alveolar) organoids, the method comprising:
a) dispensing respiratory organoids and blood vessel organoids in a well;
b) gelating the respiratory organoids and blood vessel organoids of step a); and
c) culturing and fusing the gelated respiratory organoids and blood vessel organoids of step b) in a medium for blood vessel organoid maturation.

The term “organoid” used in the present disclosure refers to a three-dimensional cell aggregate formed through self-renewal and self-organization from adult stem cells (ASC), embryonic stem cells (ESC), induced pluripotent stem cells (iPSC), etc. and may comprise an organoid or cell cluster formed from suspension cell culture. The organoid may also be called a small organ-like organ, an organ analog, or an organ-like organ. The organoid specifically comprises one or more cell types among several types of cells constituting an organ or tissue, and should be able to reproduce the form and function of the tissue or organ. In addition, by reaggregating and recombining cells using 3D culture to make them similar to the living environment, the limitations of 2D cell lines cultured using 2D culture can be overcome and the physiological functions of living organisms can be similarly reproduced, which can be applied to disease modeling, drug screening, etc.

In the present disclosure, “blood vessel organoid” is an artificial organ that represents a vascular network, such as perivascular cells, and may refer to an artificial blood vessel that can perform the same function as a blood vessel. According to an embodiment of the present disclosure, the blood vessel organoids may be produced from induced pluripotent stem cells (iPSC), without being limited thereto. In addition, the induced pluripotent stem cells may be derived from any one or more cells selected from the group consisting of umbilical cord blood, alveolar epithelial cells, and basal cells, without being limited thereto.

In the present disclosure, the blood vessel organoids, derived from induced pluripotent stem cells, may be produced according to the following stages, without being limited thereto:
1) culturing the differentiated induced pluripotent stem cells in an aggregate formation medium to aggregate them; (stage 1)
2) culturing the aggregated induced pluripotent stem cells in mesoderm induction medium to induce mesoderm differentiation; (stage 2)
3) culturing the pluripotent stem cells induced into mesoderm differentiation in a vascular induction medium to induce vascular differentiation; (stage 3)
4) culturing the pluripotent stem cells induced into vascular differentiation in a maturation medium to induce maturation of blood vessel organoids (stages 4 and 5).

In the present disclosure, to produce blood vessel organoids, STEMdiff™ Blood Vessel Organoid Kit was used, without being limited thereto.

The “induced pluripotent stem cells (iPSC)” used in the present disclosure are cells induced by artificially performing a retrodifferentiation process (reprogramming) on adult cells that have already completed differentiation, and have pluripotency. The induced pluripotent stem cells can be differentiated into cells of various organs such as the brain and heart. According to an embodiment of the present disclosure, the induced pluripotent stem cells may be induced pluripotent stem cells derived from umbilical cord blood or induced pluripotent stem cells derived from alveolar epithelial cells, without being limited thereto.

The term “somatic (=adult) cells” used in the present disclosure is a term opposite to embryonic cells and refers to cells derived from adult organisms that survive after birth. According to an embodiment of the present disclosure, the somatic cells may be umbilical cord blood or alveolar epithelial cells and the induced pluripotent stem cells of the present disclosure may be derived from umbilical cord blood or alveolar epithelial cells, without being limited thereto.

According to an embodiment of the present disclosure, blood vessel organoids may express one or more of CD31 (cluster of differentiation 31), or SMA (smooth muscle actin). In an embodiment of the present disclosure, blood vessel organoids produced from induced pluripotent stem cells expressed both CD31 and SMA, without being limited thereto.

In the present disclosure, “respiratory organoid” may refer to an artificial organ that exhibits the function of the respiratory tract (=airway). In an embodiment of the present disclosure, the respiratory organoids may be one of alveolar organoids and airway organoids (=respiratory tract organoids), without being limited thereto.

The present disclosure provides a method of producing alveolar organoids, the method comprising the following steps:
1) isolating alveolar epithelial cells from isolated alveolar epithelial tissue; and
2) culturing the alveolar epithelial cells to produce alveolar organoids.

In the present disclosure, the alveolar epithelial cells may be cultured using SAGM medium or Alveolar differentiation medium, without being limited thereto.

In the present disclosure, as the SAGM medium, SAGM™ Small Airway Epithelial Cell Growth Medium BulletKit™ (Catalog #: CC-3118) was used. This kit comprises 1×SABM™ Basal Medium (CC-3119, 500mL) and 1×SAGM™ SingleQuots™ Supplement Pack (CC-4124). In addition, the following ingredients, without being limited to, may be comprised: (1) 1×Orange Cap Vial with BPE, 2.0 mL; (2) 1×Lilac Cap Vial with Insulin, 0.5 mL; (3) 1×Natural Cap Vial with Hydrocortisone, 0.5 mL; (4) 1×Red Cap Vial with GA-1000, 0.5 mL; (5) 1×Amber Vial with Retinoic Acid, 0.5 mL; (6) 1×Bottle with Fatty Acid Free BSA, 5 mL; (7) 1×Natural Cap Vial with Transferrin, 0.5 mL; (8) 1×Amber Vial with Triiodothyronine, 0.5 mL; (9) 1×Amber Vial with Epinephrine, 0.5 mL; (10) 1×Green Cap Vial with hEGF, 0.5 mL.

In the present disclosure, the ingredients of Alveolar differentiation medium may comprise 8-Br-CAMP, FGF7 (human KGF), IBMX, B27 supplement, Dexamethasone, ITS premix, BSA, CaCl₂, Hepes, Penicillin/Streptomycin, and Ham's F12, without being limited thereto.

The present disclosure provides a method of producing airway organoids, the method comprising the following steps:
1) culturing basal cells, isolated from inferior turbinate tissue, in spheroid formation medium; and
2) culturing the spheroids, formed in the medium, in organoid differentiation medium.

In the present disclosure, spheroid formation medium may comprise a solution of BEBM and DMEM mixed in a ratio of 1:1; and BEBM excluding RA and T3, without being limited thereto.

In the present disclosure, BEBM may be a basal medium for bronchial epithelial cell growth.

In the present disclosure, DMEM is a basic medium used to support the growth of mammalian cells, and may comprise high glucose and L-glutamine, and additionally phenol red, without being limited thereto.

In the present disclosure, the spheroids may be cultured until they reach a size of 115 μm (±6.4), without being limited thereto. For examples, the size of the spheroids may be 100 μm to 130 μm, 100 μm to 128 μm, 100 μm to 126 μm, 100 μm to 125 μm, 100 μm to 124 μm, 100 μm to 121.4 μm, 103 μm to 130 μm, 103 μm to 128 μm, 103 μm to 126 μm, 103 μm to 125 μm, 103 μm to 124 μm, 103 μm to 121.4 μm, 106 μm to 130 μm, 106 μm to 128 μm, 106 μm to 126 μm, 106 μm to 125 μm, 106 μm to 124 μm, 106 μm to 121.4 μm, 108.9 μm to 130 μm, 108.9 μm to 128 μm, 108.9 μm to 126 μm, 108.9 μm to 125 μm, 108.9 μm to 124 μm, or 108.9 μm to 121.4 μm, without being limited thereto.

In the present disclosure, organoid differentiation medium may be a serum-free BPE medium for culturing human airway epithelial cells at the air-liquid interface (ALI), without being limited thereto. The medium may be PneumaCult™-ALI Medium (Catalog #05001), and may comprise PneumaCult™-ALI Basal Medium, PneumaCult™-ALI 10X Supplement, and PneumaCult™-ALI Maintenance Supplement, without being limited thereto.

In an embodiment of the present disclosure, when the respiratory organoids are airway organoids, the production method thereof may further comprise:
(S0) stabilizing airway organoids and blood vessel organoids in a medium for blood vessel organoid maturation, without being limited thereto.

In the present disclosure, the stabilization step may be performed at 20° C. to 40° C. for 12 hours to 48 hours. For examples, 20° C. to 39° C., 20° C. to 38° C., 20° C. to 37° C., 25° C. to 39° C., 25° C. to 38° C., 25° C. to 37° C., 30° C. to 39° C., 30° C. to 38° C., 30° C. to 37° C., 32° C. to 39° C., 32° C. to 38° C., 32° C. to 37° C., 34° C. to 39° C., 34° C. to 38° C., 34° C. to 37° C., 35° C. to 39° C., 35° C. to 38° C., 35° C. to 37° C., 36° C. to 39° C., 36° C. to 38° C., 36° C. to 37° C., or 37° C., without being limited thereto.

In addition, In the present disclosure, the stabilization step may be performed for 12 hours to 42 hours, 12 hours to 36 hours, 12 hours to 30 hours, 12 hours to 24 hours, 18 hours to 42 hours, 18 hours to 36 hours, 18 hours to 30 hours, 18 hours to 24 hours, 20 hours to 42 hours, 20 hours to 36 hours, 20 hours to 30 hours, 20 hours to 24 hours, 22 hours to 42 hours, 22 hours to 36 hours, 22 hours to 30 hours, 22 hours to 24 hours, 23 hours to 42 hours, 23 hours to 36 hours, 23 hours to 30 hours, 23 hours to 24 hours, or 24 hours, without being limited thereto.

In the present disclosure, the respiratory organoids may be separated from a Matrigel dome according to the following steps, but being limited to:
1) centrifuging a tube comprising respiratory organoids at 300 to 1000 rpm, 300 to 800 rpm, 300 to 600 rpm, 400 to 600 rpm, 400 to 500 rpm, or 500 rpm; and/or
for 30 seconds to 5 minutes, 30 seconds to 4 minutes, 30 seconds to 3 minutes, 30 seconds to 2 minutes, 40 seconds to 2 minutes, 50 seconds to 2 minutes, 50 seconds to 1 minute 30 seconds, 50 seconds to 1 minute, or 1 minute;
2) after the centrifugation of step 1), removing a supernatant, adding PBS and pipetting it, followed by centrifuging at 300 to 1000 rpm, 300 to 800 rpm, 300 to 600 rpm, 400 to 600 rpm, 400 to 500 rpm, or 500 rpm; and/or
for 30 seconds to 5 minutes, 30 seconds to 4 minutes, 30 seconds to 3 minutes, 30 seconds to 2 minutes, 40 seconds to 2 minutes, 50 seconds to 2 minutes, 50 seconds to 1 minute 30 seconds, 50 seconds to 1 minute, or 1 minute;
3) after the centrifugation of step 2), removing a supernatant, adding VitroGel Cell Recovery Solution and allowing to react in CO₂ incubator at 30 to 40° C., 32 to 40° C., 34 to 40° C., 36 to 40° C., 36 to 39° C., 36 to 38° C., 36 to 37° C., or 37° C.; and/or
for 10 to 20 minutes, 12 to 20 minutes, 14 to 20 minutes, 14 to 18 minutes, 14 to 16 minutes, 15 to 16 minutes, or 15 minutes;
4) after the reaction of step 3), centrifuging at 300 to 1000 rpm, 300 to 800 rpm, 300 to 600 rpm, 400 to 600 rpm, 400 to 500 rpm, or 500 rpm; and/or
for 30 seconds to 5 minutes, 30 seconds to 4 minutes, 30 seconds to 3 minutes, 30 seconds to 2 minutes, 40 seconds to 2 minutes, 50 seconds to 2 minutes, 50 seconds to 1 minute 30 seconds, 50 seconds to 1 minute, or 1 minute; and
5) after the centrifugation of step 4), removing a supernatant, adding PBS and pipetting, followed by centrifuging at 300 to 1000 rpm, 300 to 800 rpm, 300 to 600 rpm, 400 to 600 rpm, 400 to 500 rpm, or 500 rpm for 30 seconds to 5 minutes, 30 seconds to 4 minutes, 30 seconds to 3 minutes, 30 seconds to 2 minutes, 40 seconds to 2 minutes, 50 seconds to 2 minutes, 50 seconds to 1 minute 30 seconds, 50 seconds to 1 minute, or 1 minute.

Therefore, the respiratory organoids may be isolated by repeating the process of centrifuging at 500 rpm for 1 minute 2 to 10 times, 2 to 8 times, 2 to 6 times, 3 to 6 times, 4 to 6 times, 4 to 5 times, or 4 times according to an embodiment of the present disclosure, without being limited thereto.

In an embodiment of the present disclosure, a ratio of respiratory organoids: blood vessel organoids in step (S1) may be 1: (1 to 5), without being limited thereto.

In the present disclosure, in step a), respiratory organoids and blood vessel organoids may be dispensed in a ratio of 1:1 to 5, 1:1 to 4, 1:1 to 3, 1:1 to 2, or 1:1, without being limited thereto. That is, in the present disclosure, there is a possibility of fusion between blood vessels and blood vessels or between the respiratory tract and the respiratory tract when fusing multiple organoids, so each organoid was fused one by one to check functionality such as markers and morphological characteristics, without being limited thereto. Vascularized organoids may be formed by dispensing one or more airway organoids to one blood vessel organoid, or by dispensing at the opposite dispensing ratio.

In an embodiment of the present disclosure, step (S1) may be performed at 30° C. to 40° C. for 10 minutes to 2 hours, without being limited thereto. For example, step (S1) may be performed at 20° C. to 40° C. for 12 hours to 48 hours. For example, step (S1) may be performed at 20° C. to 39° C., 20° C. to 38° C., 20° C. to 37° C., 25° C. to 39° C., 25° C. to 38° C., 25° C. to 37° C., 30° C. to 39° C., 30° C. to 38° C., 30° C. to 37° C., 32° C. to 39° C., 32° C. to 38° C., 32° C. to 37° C., 34° C. to 39° C., 34° C. to 38° C., 34° C. to 37° C., 35° C. to 39° C., 35° C. to 38° C., 35° C. to 37° C., 36° C. to 39° C., 36° C. to 38° C., 36° C. to 37° C., or 37° C., without being limited thereto.

In addition, step (S1) may be performed for, for example, 10 minutes to 1 hour 40 minutes, 10 minutes to 1 hour 20 minutes, 10 minutes to 1 hour 10 minutes, 10 minutes to 1 hour, 20 minutes to 1 hour 40 minutes, 20 minutes to 1 hour 20 minutes, 20 minutes to 1 hour 10 minutes, 20 minutes to 1 hour, 40 minutes to 1 hour 40 minutes, 40 minutes to 1 hour 20 minutes, 40 minutes to 1 hour 10 minutes, 40 minutes to 1 hour, 50 minutes to 1 hour 40 minutes, 50 minutes to 1 hour 20 minutes, 50 minutes to 1 hour 10 minutes, 50 minutes to 1 hour, or 1 hour, without being limited thereto.

In the present disclosure, in step c), Matrigel may be fed into a well to 30 to 80%, 40 to 80%, 50 to 80%, 60 to 80%, 60 to 70%, 60 to 67%, 60 to 65%, 60 to 64%, 60 to 62%, 50 to 62%, 53 to 62%, 55 to 62%, 56 to 62%, 58 to 62%, 59 to 61%, 60%, 61%, 62%, 63%, 64%, or 65% of the well capacity, and may be allowed to be gelated at 30 to 40° C., 33 to 40° C., 35 to 40° C., 36 to 40° C., 37 to 40° C., 37 to 39° C., 37 to 38° C., 31° C., 32° C., 33° C., 34° C., 35° C., 36° C., 37° C., or 38° C. for 10 minutes to 2 hours, 10 minutes to 1 hour 30 minutes, 10 minutes to 1 hour 10 minutes, 10 minutes to 1 hour, 20 minutes to 1 hour, or 30 minutes to 1 hour. According to an embodiment of the present disclosure, in step c), Matrigel may be fed into a well to 60% of the well capacity and allowed to be gelated at 37° C. for 30 minutes to 1 hour, without being limited thereto.

In an embodiment of the present disclosure, in step (S2), culturing may be performed for 1 day to 15 days, without being limited thereto. For example, the culturing may be performed for 1 day to 12 days, 1 day to 9 days, 1 day to 8 days, 1 day to 7 days, 2 days to 15 days, 2 days to 14 days, 2 days to 13 days, 2 days to 12 days, 2 days to 11 days, 2 days to 10 days, 2 days to 9 days, 2 days to 8 days, or 2 days to 7 days, without being limited thereto.

In the present disclosure, in step d), culturing may be performed for 2 to 15 days, 2 to 13 days, 2 to 11 days, 2 to 10 days, 2 to 9 days, 2 to 8 days, 2 to 7 days, 2 to 6 days, 2 to 5 days, or 2 to 4 days. According to an embodiment of the present disclosure, the culturing may be performed for 2 to 7 days, but being not limited to, and may be appropriately adjusted depending upon the size of organoids and the distance between organoids to be fused.

In the present disclosure, the “medium for blood vessel organoid maturation” may refer to a medium capable of maturing blood vessel organoids. In addition, the medium for maturing blood vessel organoids of the present disclosure may refer to any cell culture medium comprising the following ingredients: p160ROCK inhibitor, an inhibitor of GSK-3 enzyme, bone morphogenetic protein 4, a vascular endothelial growth factor and a fibroblast growth factor. In addition to the ingredients, the culture medium may comprise a number of ingredients necessary for the function of cultured cells, for example, a buffer solution, an antibiotic and/or commercialized medium additives, etc., without being limited thereto.

In an embodiment of the present disclosure, STEMdiff™ Blood Vessel Organoid Maturation Medium is used as the medium for blood vessel organoid maturation, but without being limited thereto, any medium capable of maturing blood vessel organoids may be used without limitation.

The kit was manufactured with reference to DOI: 10.1038/s41596-019-0213-z. In an embodiment of the present disclosure, Y-27632, CHIR99021, BMP-4m, VEGF-A, forskolin, 15% FBS, VEGF-A and FGF-2 may be comprised, without being limited thereto.

In the present disclosure, “culture” refers to any actions performed to grow cells under appropriately artificially controlled environmental conditions.

The present disclosure provides vascularized respiratory organoids produced by any one of the above methods.

In the present disclosure, “vascularized respiratory organoids” may refer to respiratory (airway and alveolar) organoids in which a vascular network is created by fusion with blood vessel organoids to induce vascularization.

In an embodiment of the present disclosure, the respiratory organoids may be one of alveolar organoids and airway organoids, without being limited thereto.

In an embodiment of the present disclosure, the vascularized respiratory organoids may have a structure wherein blood vessel organoids surround respiratory organoids, without being limited thereto.

In an embodiment of the present disclosure, the vascularized respiratory organoids may exhibit a morphological feature wherein new blood vessels are generated and extended, without being limited thereto.

In the present disclosure, respiratory organoids may be vascularized in a form in which blood vessel organoids surround respiratory organoids. Accordingly, it has been proven that a vascular network is formed in respiratory organoids, making it possible to produce respiratory organoids that more precisely mimic the actual situation in the human body.

In addition, such a “vascularized” feature was re-proven through markers.

In an embodiment of the present disclosure, when the vascularized respiratory organoids are vascularized alveolar organoids, the vascularized alveolar organoids may express one or more of CD31 (cluster of differentiation 31) and SMA (smooth muscle actin), without being limited thereto.

In addition, according to an embodiment of the present disclosure, the vascularized respiratory organoids may express one or more of CD31 (cluster of differentiation 31) and tubulin, without being limited thereto, when the vascularized respiratory organoids are vascularized airway organoids.

According to an embodiment of the present disclosure, since a vascular network is generated in the vascularized respiratory (airway and alveolar) organoids, the vascularized respiratory (airway and alveolar) organoids are characterized by expressing blood vessel-related markers CD31 (cluster of differentiation 31) and SMA (smooth muscle actin).

In addition, according to an embodiment of the present disclosure, blood vessel organoids in the vascularized respiratory (airway and alveolar) organoids may surround respiratory (airway and alveolar) organoids, without being limited thereto.

In the present disclosure, “CD31 (cluster of differentiation 31)”, also known as platelet endothelial cell adhesion molecule (PECAM-1), is a vascular endothelial cell-specific marker and is one of human proteins encoded by the PECAM1 gene present on chromosome 17. It is known to play a role in removing aged neutrophils in the human body. It is involved in intercellular connections of endothelial cells, and is also expressed in neutrophils, monocytes, natural killer cells, hematopoietic stem cells, and certain subpopulations of lymphocytes.

The term “SMA (smooth muscle actin) used in the present disclosure, known as α-smooth muscle actin (α-SMA), α-actin-2, SMactin, or ASMA, is a protein expressed by the ACTA2 gene, and is a smooth muscle cell-specific marker.

In addition, the present disclosure provides a vascularized respiratory (airway and alveolar) organoid chip comprising an organoid culture chamber loaded with the vascularized respiratory (airway and alveolar) organoids according to the present disclosure.

The term “organoid chip” used in the present disclosure is a hybrid device with the form of a conventional semiconductor chip made by combining organoids and inorganic materials such as semiconductors or glass. By utilizing the unique functions of biomolecules and mimicking the functions of living organisms, it has the characteristic of diagnosing infectious diseases, analyzing genes, and serving as a new functional device for new information processing. In addition, the organoid chip comprises a biosensor that can detect and analyze various biochemical substances, such as a lab on a chip with an automatic analysis function, by compactly integrating sample pretreatment, biochemical reaction, detection, and data analysis, and can be broadly defined.

The term “organoid culture chamber” used in the present disclosure refers to a space for generating or maintaining organoids. For example, it may be a space for differentiating cells or cells isolated from a specific tissue into tissue or organ cells with a specific function, and/or may be a space for surviving, growing, or proliferating organoids.

According to an embodiment of the present disclosure, the organoid culture chamber may comprise a medium for blood vessel organoid maturation to induce vascularization of vascularized airway organoids, without being limited thereto.

The present disclosure provides a kit for producing vascularized respiratory organoids, comprising (a) a composition for producing blood vessel organoids and (b) a composition for producing respiratory organoids,
wherein each of the compositions comprises any one selected from a group consisting of the following components:
(a) umbilical cord blood, alveolar epithelial cells, and basal cells; and
(b) alveolar epithelial tissue, and basal cells.

The kit of the present disclosure may further comprise an instruction. The instruction may comprise the production method, without being limited thereto.

In the present disclosure, the “kit” refers to a tool for manufacturing blood vessel organoids, respiratory organoids, and even vascularized respiratory organoids using the composition of the present disclosure. In addition to the above materials, the kit of the present disclosure may comprise other components, compositions, solutions, devices, etc. commonly required for their storage and disposal methods. As a specific example, each composition may be applied without limit, more than 1 time, there is no limit on the order of application of each material, and the application of each material may be carried out simultaneously or at a microscopic level.

In the present disclosure, the kit may comprise a container; an instruction; etc. The container can serve to package the materials, as well as store and secure them. The container may take the form of, for example, a bottle, a tub, a sachet, an envelope, a tube, an ampoule, etc., which may be partially or wholly plastic, glass, paper, foil, etc. The container may be equipped with a fully or partially separable stopper, which may initially be part of the container or attached to the container by mechanical, adhesive, or other means. The container may also be equipped with a stopper, which may be accessible to the contents by a needle. The kit may comprise an external package, which may comprise an instruction for use of the components.

In addition, the present disclosure provides vascularized respiratory (airway and alveolar) organoids produced by the method of producing the vascularized respiratory (airway and alveolar) organoids according to the present disclosure.

In the present disclosure, the vascularized respiratory (airway and alveolar) organoids can be used to treat subjects in vivo. The present disclosure comprises a method of subject's respiratory tract diseases. As disclosed in the present disclosure, one method involves ameliorating, treating, or alleviating the symptoms of a respiratory disease in a subject in need thereof. The method also comprises administering the vascularized respiratory (airway and alveolar) organoids in an amount effective for treating respiratory disease of the subject.

The method of treating a subject may further comprise administering vascularized respiratory (airway and alveolar) organoids or transplanting vascularized respiratory (airway and alveolar) organoids. As a result of administration or transplantation, the symptoms of respiratory disease in the subject are improved, cured, or alleviated. The improvement, treatment or alleviation may be any change in the respiratory disease or symptoms of the respiratory disease that can be detected using natural senses or artificial devices.

A “subject” or “patient” as used in the present disclosure may be a human or non-human mammal. A non-human mammal comprises, for example, livestock and pets, such as sheep, cattle, pigs, dogs, cats and murine mammals. Preferably, the subject is a human. A respiratory disease that can be treated using the method of the present disclosure is a physically manifest disease or condition related to the respiratory system, and is, for example, cystic fibrosis, respiratory distress syndrome, acute respiratory distress syndrome, pulmonary tuberculosis, cough, bronchial asthma, cough based on increased airway hyperreactivity (bronchitis, influenza syndrome, asthma, obstructive pulmonary disease, etc.), flu syndrome, cough suppression, airway hyperresponsiveness, tuberculosis disease, asthma (airway inflammatory cell infiltration, increased airway hyperresponsiveness, bronchoconstriction, mucus hypersecretion, etc.), chronic obstructive pulmonary disease, emphysema, pulmonary fibrosis, idiopathic pulmonary fibrosis, reversible airway obstruction, adult respiratory disease syndrome, pigeon breeder's disease, farmer's lung, bronchopulmonary dysplasia, airway disease, emphysema, allergic bronchopulmonary aspergillus, allergic bronchitis bronchiectasis, occupational asthma, reactive airway disease syndrome, interstitial lung disease, parasitic lung disease, etc., without being limited thereto.

The vascularized respiratory (airway and alveolar) organoids of the present disclosure may be comprised in an amount of 1 pg to 30 g w/v % in a pharmaceutical composition, without being limited thereto.

In the present disclosure, “individual” is not limited so long as it is a vertebrate, but may be specifically a human, a mouse, a rat, a guinea pig, a rabbit, a monkey, a pig, a horse, a cow, a sheep, an antelope, a dog and a cat, and is preferably a human.

In the present disclosure, “administration” means introducing a pharmaceutical composition of the present disclosure to a patient by any appropriate method, and the route of administration of the composition of the present disclosure may be various routes, such as oral or parenteral, so long as it can reach a target tissue.

In the present disclosure, “prevention” means any action that delays respiratory disease by administering a composition according to the present disclosure, “treatment” means any action in which respiratory disease symptoms are improved or beneficially changed by the administration of a pharmaceutical composition according to the present disclosure, and “improvement” means any action that reduces parameters related to a respiratory tract disease, such as the severity of symptoms, by administering a composition according to the present disclosure.

In the present disclosure, a pharmaceutical composition may further comprise a suitable carrier, excipient, and diluent commonly used in the manufacture of a pharmaceutical composition.

In the present disclosure, “carrier” is also called a vehicle and refers to a compound that facilitates the addition of proteins or peptides into cells or tissues. For example, dimethyl sulfoxide (DMSO) is a commonly used carrier that facilitates the introduction of many organic substances into cells or tissues of living organisms

In the present disclosure, “diluent” is defined as a compound that not only stabilizes the biologically active form of the protein or peptide of interest, but also that is used to dilute water in which the protein or peptide is dissolved. A salt dissolved in a buffer solution is used as a diluent in the art. A commonly used buffer solution is phosphate-buffered saline because it mimics the salt state of human body fluids. Since buffer salt can control the pH of a solution at low concentrations, it is rare for buffer diluents to modify the biological activity of compounds. As used herein, compounds comprising azelaic acid can be administered to human patients by themselves, with other ingredients as in combination therapy, or as a pharmaceutical composition mixed with suitable carriers or excipients.

In addition, a pharmaceutical composition according to the present disclosure maybe formulated for external application such as powders, granules, tablets, capsules, suspensions, emulsions, syrups, or aerosols or in the form of a sterile injectable solution, examples of a carrier, excipient and diluent that can be comprised in the composition comprise lactose, dextrose, sucrose, oligosaccharide, sorbitol, mannitol, xylitol, erythritol, maltitol, starches, gum acacia, alginate, gelatin, calcium phosphate, calcium silicate, cellulose, methyl cellulose, microcrystalline cellulose, polyvinylpyrrolidone, water, methyl hydroxybenzoate, propyl hydroxybenzoate, talc, magnesium stearate, and mineral oil. In the case of the formulations, the formulation may be prepared by using a diluent or an excipient, such as a filler, an extender, a binder, a wetting agent, a disintegrating agent, and a surfactant which are generally used. Solid formulations for oral administration comprise tablets, pills, powders, granules, capsules, and the like, and the solid formulation may be prepared by mixing at least one excipient, for example, starch, calcium carbonate, sucrose or lactose, gelatin, and the like. In addition, lubricants such as magnesium stearate, talc may be used in addition to simple excipients. Liquid formulations for oral administration may correspond to suspensions, oral liquids, emulsions, syrups, and the like, and may comprise various excipients, for example, a wetting agent, a sweetener, an aromatic agent, a preservative, and the like, in addition to water and liquid paraffin which are commonly used as simple diluents. Formulations for parenteral administration comprise sterilized aqueous solutions, non-aqueous solvents, suspensions, emulsions, lyophilized agents, and suppositories. As the non-aqueous solution and the suspension, propylene glycol, polyethylene glycol, vegetable oil such as olive oil, injectable ester such as ethyl oleate, and the like may be used. As a base of the suppository, witepsol, macrogol, tween 61, cacao butter, laurinum, glycerogelatin, and the like may be used.

The pharmaceutical composition of the present disclosure can be administered orally or parenterally, preferably parenterally, and in the case of parenteral administration, it may be administered by intramuscular injection, intravenous injection, subcutaneous injection, intraperitoneal injection, topical administration, transdermal administration, etc.

The appropriate dosage of the pharmaceutical composition of the present disclosure may vary depending on factors such as formulation method, administration method, patient's age, weight, sex, pathological condition, diet, administration time, administration route, excretion rate and reaction sensitivity.

The pharmaceutical composition of the present disclosure may be formulated in unit dosage form using a pharmaceutically acceptable carrier and/or excipient according to a method that can be easily performed by a person skilled in the art to which the disclosure pertains, or may be manufactured by placing it in a bulk container. Here, the formulation may be in the form of a solution, suspension or emulsion in an oil or aqueous medium, or may be in the form of an extract, powder, granule, tablet or capsule, and may additionally comprise a dispersant or a stabilizer.

In this specification, “active ingredient” refers to an ingredient that can exhibit the desired activity alone or together with a carrier that is inactive on its own.

Most of the terms used herein are general terms that have been widely used in the technical art to which the present disclosure pertains. However, some of the terms used herein may be created reflecting intentions of technicians in this art, precedents, or new technologies. Also, some of the terms used herein may be arbitrarily chosen by the present applicant. In this case, these terms are defined in detail below. Accordingly, the specific terms used herein should be understood based on the unique meanings thereof and the whole context of the present disclosure.

The terms such as “first” and “second” are used herein merely to describe a variety of constituent elements, but the constituent elements are not limited by the terms. The terms are used only for the purpose of distinguishing one constituent element from another constituent element. For example, a first element may be termed a second element and a second element may be termed a first element without departing from the scope of rights according to the concept of the present disclosure. The term “and/or” comprises any or all combinations of one or more of the associated listed items.

Throughout the specification of the present disclosure, when a part is said to “comprise” a certain component, this means that it may further comprise other components rather than excluding other components unless specifically stated to the contrary. The terms “about,” “substantially,” and the like used in this specification are used to mean at or close to a presented numerical value when manufacturing and material tolerances inherent in stated meaning are presented, and are used to prevent unscrupulous infringers from unfairly exploiting the disclosure in which precise or absolute figures are mentioned to aid understanding of the embodiments.

Throughout this specification, the term “a combination thereof” comprised in the Markushi format expression refers to a mixture or combination of one or more elements selected from the group consisting of components described in the Markushi format expression, and to comprise one or more selected from the group consisting of the components.

Now, the present disclosure will be described in more detail with reference to the following preferred examples. These examples are provided for illustrative purposes only and should not be construed as limiting the scope and spirit of the present disclosure.

### EXAMPLES

### Example 1. Production of Vascular Organoids and Analysis of Characterization of Vascular Organoids

### Example 1-1. Production of Blood Vessel Organoids Using Induced Pluripotent Stem Cells Derived From Basal Cells and Alveolar Epithelial Cells

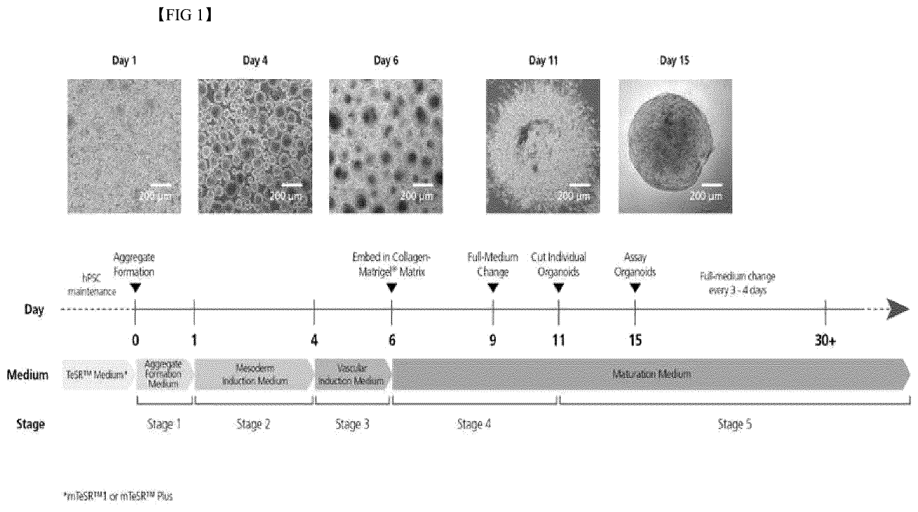

Blood vessel organoids were produced using induced pluripotent stem cells derived from basal cells and alveolar epithelial cells. Specifically, blood vessel organoids were produced using STEMdiff™ Blood Vessel Organoid Kit (stemcell_Catalog #100-0651) while replacing a medium through five steps according to the manufacturer's protocol shown in FIG. 1. The produced blood vessel organoids were cultured in a 96-well plate until fused with organoids.

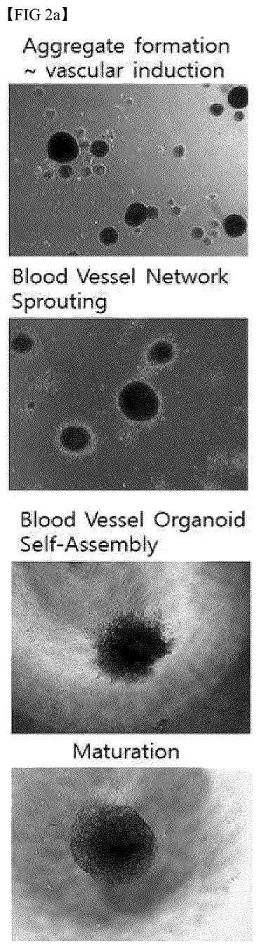

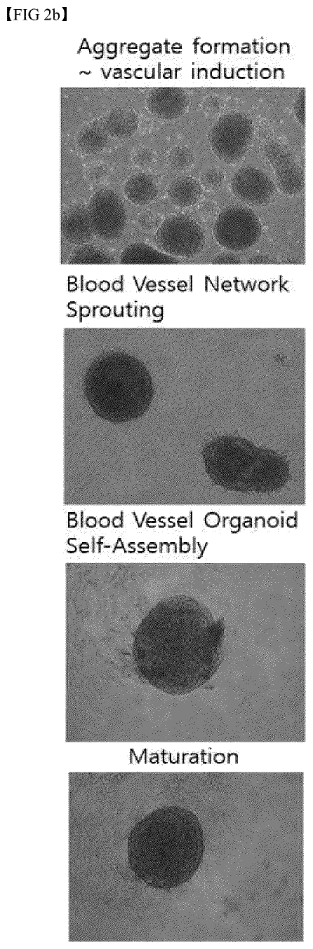

While blood vessel organoids were produced using induced pluripotent stem cells derived from basal cells and alveolar epithelial cells, the formation process of blood vessel organoids was observed under a microscope, and results are shown in FIGS. 2A and FIG. 2B, respectively.

### Example 1-2. Analysis of Characteristics of Blood Vessel Organoids

The characteristics of the blood vessel organoids produced using induced pluripotent stem cells derived from basal cells and alveolar epithelial cells of Example 1-1 were analyzed. Specifically, based on the previously reported information (Nat Protoc 14, 3082-3100 (2019)), each blood vessel organoid was fluorescently stained for CD31 (cluster of differentiation 31) and SMA (smooth muscle actin) which are specific factors that recognize blood vessels.

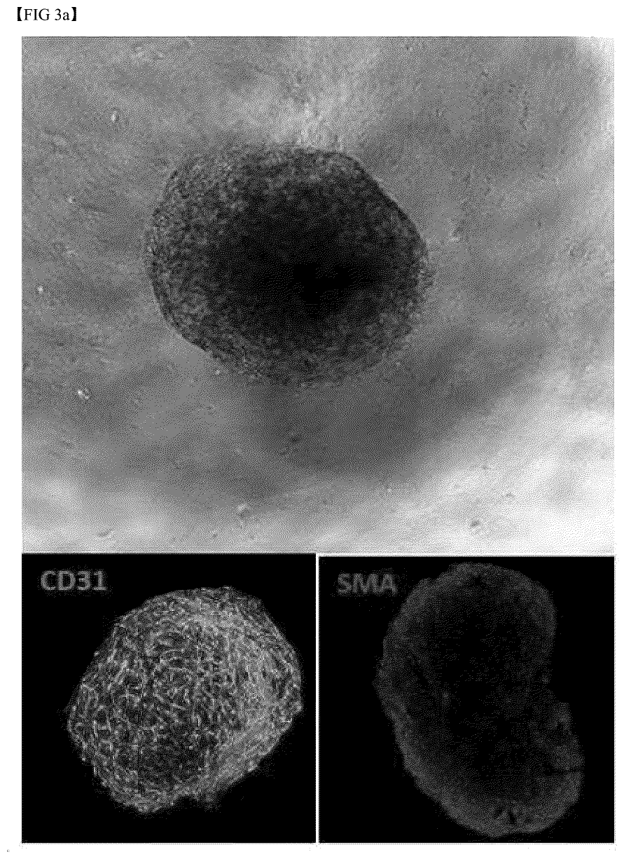

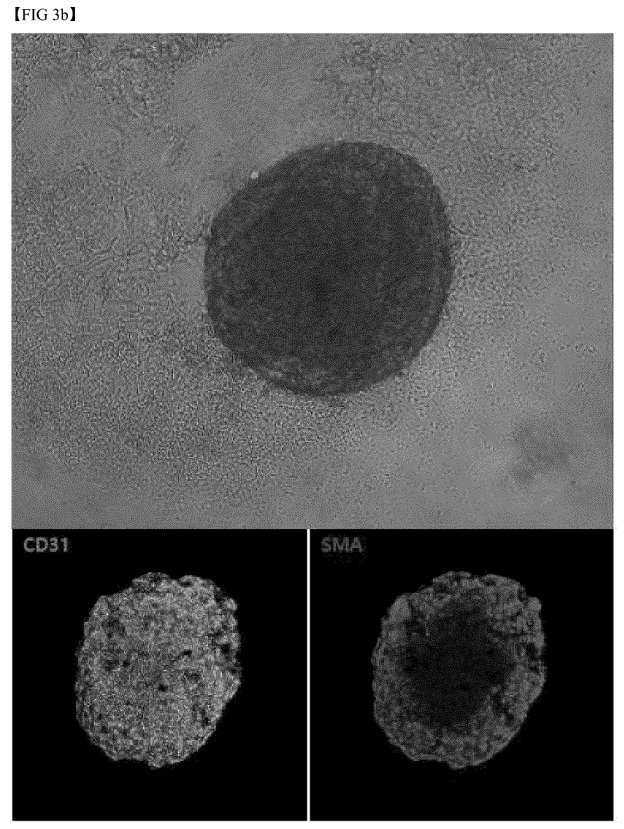

As a result, in all blood vessel organoids produced using induced pluripotent stem cells derived from basal cells and alveolar epithelial cells, the vascular markers CD31 and SMA were found to be expressed in blood vessel organoids after the maturation stage, confirming that mature blood vessel organoids were successfully produced (FIGS. 3A and 3B).

### Example 2. Production of Airway and Alveolar Organoids

Before manufacturing vascularized respiratory organoids, respiratory organoids (Airway and Alveolar organoids) to be fused with blood vessel organoids were first prepared. As respiratory organoids, alveolar organoids using alveolar epithelial cells derived from alveolar epithelial tissue, and airway organoids using basal cells were produced, respectively.

### Example 2-1. Production of Alveolar Organoids Derived From Alveolar Epithelial Tissue

First, alveolar organoids were produced using alveolar epithelial tissue. Specifically, alveolar epithelial tissue was cut into small pieces with a diameter of 1 mm³ or less, and the cut tissue was added along with collagenase, elastase, and trypsin to Hanks Balanced Salt Solution (HBSS), and incubated at 37° C. for 30 minutes. The solution comprising the tissues after incubation was filtered through a 40 μm cell strainer to remove any remaining large tissues. The solution from which large tissues were removed with a 40 μm cell strainer was centrifuged at a speed of 1500 RPM for 10 minutes, and then a supernatant was removed, leaving only the settled pellet. Red Blood Cell Lysis Buffer (cat. 11814389001, Roche) was added to the settled pellet and reacted at room temperature for 5 minutes. After 5 minutes, centrifugation was performed for 3 minutes at a speed of 1500 RPM. A supernatant was removed, leaving only the settled pellet. The settled pellet was released into single cell units using SAGM medium (SAGM™ Small

Airway Epithelial Cell Growth Medium BulletKit™, Catalog #: CC-3118) supplemented with Y-27632 and ROCK Inhibitor, and dispensed in a culture dish. Next, it was replaced with a new SAGM medium once every 2 days.

Alveolar epithelial cells isolated from alveolar epithelial tissue were separated in a culture dish using the Animal Component-Free Cell Dissociation Kit (Cat. 05426, STEMCELL). The cells separated in the culture dish were added to Matrigel (60% Matrigel+40% SAGM) in a diluted amount of 5×10³/50 ul and mixed therein. 50 ul of gel comprising cells was dispensed into a culture plate and incubated at 37° C. in a CO2 incubator for 30 minutes to cause gelation. After gelation was completed, SAGM medium was added and cultured for 7 days, and the medium was replaced with fresh medium every 2 days. After culturing in SAGM medium for 7 days, this medium was replaced with Alveolar differentiation medium, and the medium was replaced with fresh medium every 2 days. The alveolar organoids were cultured in the alveolar differentiation medium (Table 1 below) for a minimum of 21 days and a maximum of 40 days. The produced alveolar organoids were verified through immunofluorescence staining and cultured in a 24-well Matrigel dome until fusion with blood vessel organoids.

TABLE 1

| Media component | Final concentration | |
|---|---|---|
| 8-Br-CAMP | 100 | μM |
| FGF7 (human KGF) | 10 | ng/mL |
| IBMX | 100 | μM |
| B27 supplement | 1x | |
| Dexamethasone | 50 | nM |
| ITS premix | 0.10% | |
| BSA | 0.25% | |
| CaCl₂ | 0.8 | mM |
| Hepes | 15 | mM |
| Penicillin/Streptomycin | 100 U · mL⁻¹/100 vg · mL⁻¹ | |
| Ham's F12 | 20 | mL |

### Example 2-2. Production of Airway Organoids Using Basal Cells

Basal cells isolated from human inferior turbinate tissue were prepared at a concentration of 1×10³/40 μl in one well. Next, cells mixed with 60% growth factor-reduced Matrigel (corning 354230) were dispensed in an amount of 40 μl each in a 48-well non-coating plate and gelated for 20 minutes in a 37° C. cell incubator. Next, 500 μl of spheroid formation medium (Lonza, BEBM and Gibco, high glucose DMEM 1:1 mix+BEBM single quotes excluding RA and T3 added) was added and replaced with a new medium the next day. After culturing for 4-7 days in a spheroid formation medium, it was replaced with a new medium every 2 days. When the size of the spheroids reached an average of 115 μm (±6.4), the medium was replaced with an upper respiratory organoid differentiation medium (stem cell, PneumaCult™-ALI medium, catalog #5001), and was replaced with a new medium every 2 days for 28-40 days.

### Example 3. Production of Vascularized Respiratory Organoids by Fusing Blood Vessel OrganoidsWwith Respiratory (Airway and Alveolar) Organoids

The blood vessel organoids manufactured in Example 1 and the respiratory organoids manufactured in Example 2 were fused to produce vascularized respiratory organoids fused with blood vessel organoids. Here, as respiratory organoids, alveolar organoids manufactured in Example 2-1 and airway organoids manufactured in Example 2-2 were used, respectively.

### Example 3-1. Production of Vascularized Alveolar Organoids

First, vascularized respiratory organoids were produced by fusing blood vessel organoids and alveolar organoids. Specifically, the blood vessel organoids were transferred to a new EP tube using a pipette. Next, to separate the alveolar organoids cultured in a Matrigel dome, the Matrigel dome was transferred to a new EP tube and the Matrigel was removed with cold PBS. Specifically, the Matrigel dome comprising alveolar organoids was separated from the culture plate and collected in a conical tube, and then the conical tube comprising alveolar organoids was centrifuged for 1 minute at a speed of 500 RPM. After centrifugation, a supernatant was removed, cold PBS was added and pipetted, centrifugation was performed again at 500 RPM for 1 minute, and a supernatant was removed. After removing the supernatant, VitroGel Cell Recovery Solution (cat. MS03-100, TheWell Bioscience) was added and reacted at 37° C. in a CO² incubator for 15 minutes. After 15 minutes, a supernatant was removed by centrifuging for 1 minute at 500 RPM. Alveolar organoids were separated by repeating the process of adding PBS, pipetting it, centrifuging at 500 RPM for 1 minute, and removing a supernatant twice.

After transferring one alveolar organoid separated from Matrigel to an EP tube comprising one blood vessel organoid using a pipette, the alveolar organoid and the blood vessel organoid were seeded in one well of 24-well Cryschem Plate (Hampton Research #HR3-159) so that they were close to each other. Next, 60% of Matrigel was added to the well comprising organoids, and gelation was performed at 37° C. for 30 minutes to 1 hour. The Matrigel comprising alveolar organoids and blood vessel organoids were separated from the 24-well Cryschem Plate, transferred to a non-coating dish, and then STEMdiff™ Blood Vessel Organoid Maturation Medium (stemcell #100-0658) was added thereto to the extent that the Matrigel was submerged.

Since a certain amount of time is required for fusion depending on the distance between organoids or the size of the organoids, the organoids were cultured in the gel for 2 to 7 days to allow the blood vessel organoids and alveolar organoids to fuse. Since the fusion process can be observed when the distance between blood vessel organoids and alveolar organoids becomes closer, the culture was sufficiently performed until only blood vessel organoids were observed under the microscope. In the present disclosure, organoids that are a fusion of blood vessel organoids and alveolar organoids are named “vascularized alveolar organoids.”

### Example 3-2. Production of Vascularized Airway Organoids

First, vascularized respiratory organoids were produced by fusing blood vessel organoids and airway organoids. Specifically, blood vessel organoids cultured in a 96 well were transferred to a new Low Cell Attachment 96 well plate (SPL #34896) using a pipette. Next, to separate the airway organoids cultured in the Matrigel dome in the 48 well, the Matrigel dome was transferred to a new EP tube, and the Matrigel was removed with cold PBS. One airway organoid separated from Matrigel was transferred to a 96-well plate comprising a blood vessel organoid using a pipette. Next, 100 μl of STEMdiff™ Blood Vessel Organoid Maturation Medium (stemcell #100-0658) was added to the one well comprising the airway organoids and the blood vessel organoids, and then stabilized at 37° C. for one day. Next, the medium was removed, and 60% Matrigel was carefully added thereto using a pipette, resuspended together with organoids, transferred to a non-coating dish, and gelated at 37° C. for 1 hour. Finally, STEMdiff™ Blood Vessel Organoid Maturation Medium was poured to submerge the Matrigel, and cultured for about 3 to 10 days until fusion could be observed under an optical microscope. In the present disclosure, organoids that are a fusion of blood vessel organoids and airway organoids are named “vascularized airway organoids”.

### Example 3-3. Analysis of Characteristics of Fused Organoids

The characteristics of the lung-blood vessel fusion organoids (vascularized alveolar organoids) and respiratory tract-blood vessel fusion organoids (vascularized airway organoids) produced in each of Examples 3-1 and 3-2 were analyzed. Here, to confirm the characteristics of the fused organoids, they were observed through fluorescence staining using a microscope and specific factors (CD31 and SMA) that recognize blood vessels in the same manner as in Example 1.

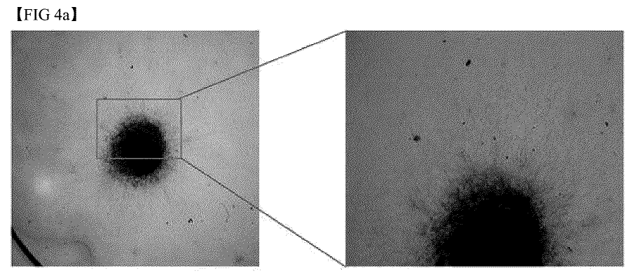

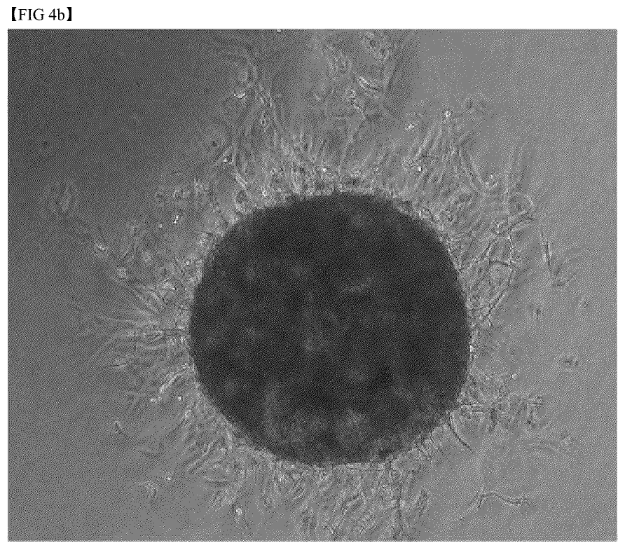

As a result of microscopic observation, it was observed that all of blood vessel organoids using alveolar epithelial cells and induced pluripotent stem cells derived from basal cells underwent gelation after the maturation stage, resulting in the creation of new blood vessels in the organoids and extending out (FIGS. 4A and 4B).

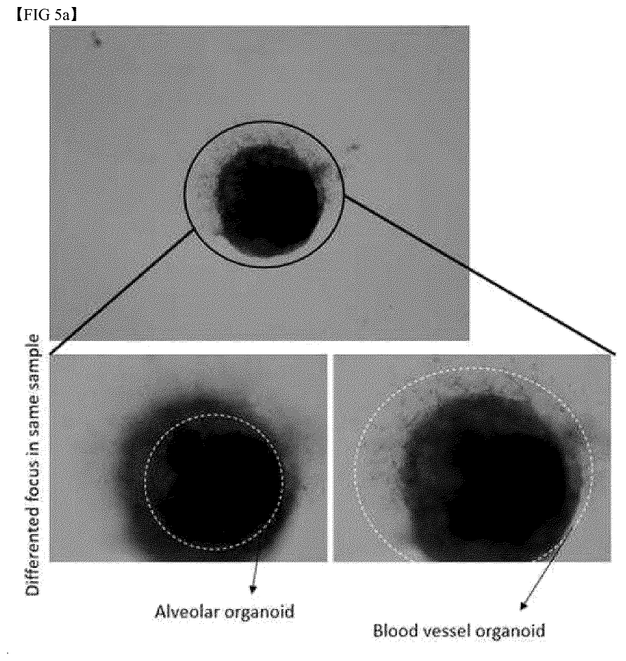

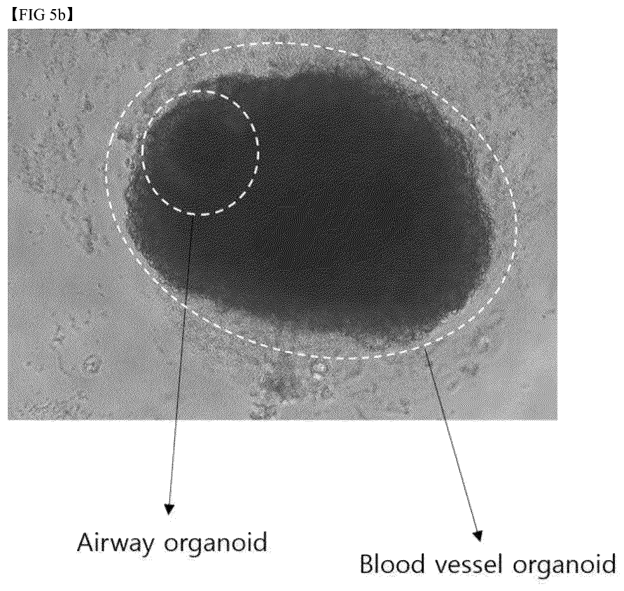

In addition, it was confirmed that in all of lung-blood vessel fusion organoids and respiratory tract-blood vessel fusion organoids, blood vessel organoids surrounded alveolar organoids (FIGS. 5A and 5B).

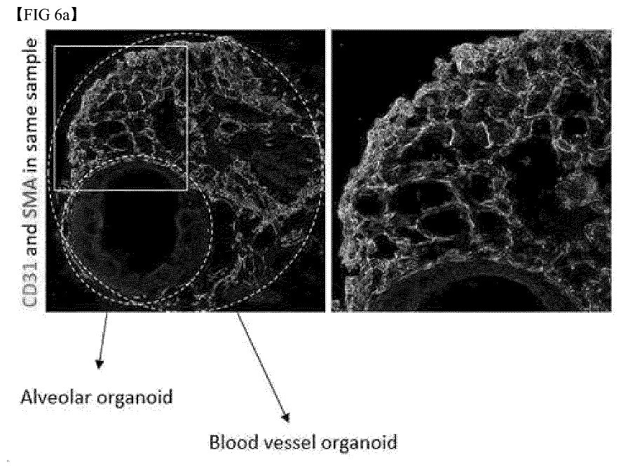

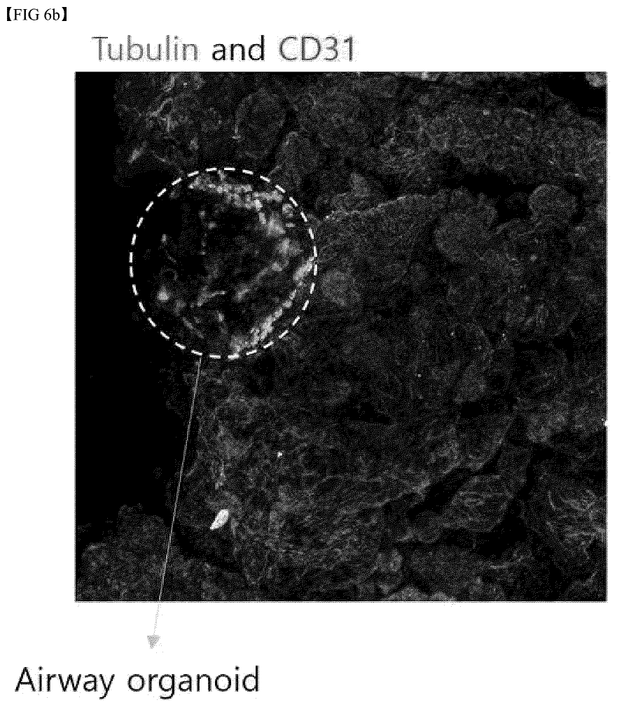

Additionally, as a result of fluorescent staining, the structural arrangement wherein blood vessel organoids surrounded alveolar organoids was reconfirmed in all of the lung-blood vessel fusion organoids and the respiratory tract-blood vessel fusion organoids (FIGS. 6A and 6B).

In addition, the lung-blood vessel fusion organoids were observed to express vascular markers CD31 and SMA. Meanwhile, the respiratory tract-blood vessel fusion organoids expressed both cilia (acetylated á tubulin), as a marker for respiratory organoids, and CD31, as a marker for blood vessel organoids, confirming that both the organoids were successfully fused.

As apparent from the above description, the present disclosure can produce vascularized respiratory (airway and alveolar) organoids with a vascular network by fusing blood vessel organoids with respiratory (airway and alveolar) organoids to induce vascularization. The produced vascularized respiratory (airway and alveolar) organoids can have very similar functions to the human lung and respiratory tract, so it is expected that they can be transplanted into humans to treat respiratory diseases or be used as an in vitro respiratory model. In addition, through the application of vascularized respiratory (airway and alveolar) organoids and vascularized chips, it can be used as a vascularized model suitable for human simulation and is expected to be applicable to drug screening.

The aforementioned description of the present disclosure is provided by way of example and those skilled in the art will understand that the present disclosure can be easily changed or modified into other specified forms without change or modification of the technical spirit or essential characteristics of the present disclosure. Therefore, it should be understood that the aforementioned examples are only provided by way of example and not provided to limit the present disclosure.

What is claimed is:

1. A method of producing a vascularized respiratory organoid, the method comprising:
(S1) gelating a blood vessel organoid and respiratory organoid dispensed into one well; and
(S2) culturing and fusing the gelated blood vessel organoid and respiratory organoid of (S1) in a medium for blood vessel organoid maturation.

2. The method according to claim 1, wherein the blood vessel organoid is derived from induced pluripotent stem cells (iPSC).

3. The method according to claim 2, wherein the induced pluripotent stem cells are derived from any one or more cells selected from the group consisting of umbilical cord blood, alveolar epithelial cells, and basal cells.

4. The method according to claim 1, wherein the blood vessel organoid expresses any one or more of CD31 (cluster of differentiation 31) and SMA (smooth muscle actin).

5. The method according to claim 1, wherein the respiratory organoid is any one of an alveolar organoid and an airway organoid.

6. The method according to claim 5, wherein the respiratory organoid is an airway organoid, and the method further comprises (S0) stabilizing an airway organoid and a blood vessel organoid in a medium for blood vessel organoid maturation.

7. The method according to claim 1, wherein, in step (S1), the ratio of the respiratory organoid: the blood vessel organoid is 1:(1 to 5).

8. The method according to claim 1, wherein step (S1) is performed at 30° C. to 40° C. for 10 minutes to 2 hours.

9. The method according to claim 1, wherein, in step (S2), the culturing is performed for 1 day to 15 days.

10. A vascularized respiratory organoid, produced by the method of claim 1.

11. The vascularized respiratory organoid according to claim 10, wherein the respiratory organoid is any one of an alveolar organoid and an airway organoid.

12. The vascularized respiratory organoid according to claim 10, wherein the vascularized respiratory organoid has a structure wherein a blood vessel organoid surrounds a respiratory organoid.

13. The vascularized respiratory organoid according to claim 10, wherein the vascularized respiratory organoid has a morphological feature wherein a new blood vessel is generated and spreads out.

14. The vascularized respiratory organoid according to claim 10, wherein the vascularized respiratory organoid is a vascularized alveolar organoid, and the vascularized alveolar organoid expresses any one or more of CD31 (cluster of differentiation 31) and SMA (smooth muscle actin).

15. The vascularized respiratory organoid according to claim 10, wherein the vascularized respiratory organoid is a vascularized airway organoid, and the vascularized respiratory organoid expresses any one or more of CD31 (cluster of differentiation 31) and tubulin.

16. A vascularized respiratory organoid chip, comprising an organoid culture chamber loaded with the vascularized respiratory organoid of claim 10.

17. The vascularized respiratory organoid chip according to claim 16, wherein the organoid culture chamber comprises a medium for blood vessel organoid maturation.
